(19) United States
(12) Patent Application Publication
(10) Pub. No.: US 2012/0100537 A1
(43) Pub. Date: Apr. 26, 2012

(54) Method of Prenatal Molecular Diagnosis of Down Syndrome and Other Trisomic Disorders

(75) Inventors: Scott A. Rivkees, Orange, CT (US); Jeffrey R. Gruen, Hamdem, CT (US); Seiyu Hosono, Rye, NY (US); Karl Hager, Branford, CT (US)

(73) Assignees: JS Genetics Inc.; Yale University

(21) Appl. No.: 13/266,429

(22) PCT Filed: Apr. 26, 2010
(86) PCT No.: PCT/US10/32426

§ 371 Date: Jan. 10, 2012

Related U.S. Application Data
(60) Provisional application No. 61/173,336, filed on Apr. 28, 2009.

(51) Int. Cl. C12Q 1/68

(52) U.S. Cl. 435/6.11

(57) ABSTRACT

The present invention encompasses a method of diagnosing chromosomal trisomy in a human subject. In one embodiment, the method comprises pyrosequencing at least one single nucleotide polymorphism on a chromosome being assessed for trisomy, where the SNP comprises two alleles.

### BACKGROUND OF THE INVENTION

A normal human karyotype is designated as 46,XX or 46,XY, indicating 46 chromosomes with an XX arrangement typical of females and 46 chromosomes with an XY arrangement typical of males, respectively. Trisomy is a form of aneuploidy with the presence of three copies of a particular ehormosome instead of the usual pair. Full trisomy of an individual usually occurs as a result of a non-disjunction event during cell division, for example, during the meiotic divisions of gametogenesis. This can result in an extra or missing chromosome (either 24 or 22 chromosomes instead of the typical 23) in a sperm or egg cell. After fertilization, the resulting fetus has 47 chromosomes instead of the typical 46. Partial trisomy occurs when part of an extra chromosome is attached to one of the other chromosomes, or if one of the chromosomes has two copies of part of its chromosome. Mosaic trisomy is a condition where extra chromosomal material exists in only some of the organism's cells and the remaining cells have the normal complement of chromosomal material.

Trisomy can occur with any chromosome and is designated either autosomal trisomy or sex-chromosome trisomy. The most common is trisomy 16 which usually results in spontaneous miscarriage in the first trimester following fertilization. The most common trisomies that survive to birth in humans are trisomy 21 (Down syndrome), trisomy 18 (Edwards syndrome), trisomy 13 (Patau syndrome), trisomy 12 (chronic lymphatic leukemia), trisomy 9, trisomy 8 (Warkany syndrome 2), Triple X syndrome (XXX), Klinefelters syndrome (XXY), and XYY syndrome.

Trisomy 21 (e.g., 47,XX,+21) is the cause of approximately 95% of observed Down syndrome, with 88% coming from nondisjunction in the maternal gamete and 8% coming from nondisjunction in the paternal gamete The effects of the extra copy vary greatly among people, depending on the extent of the extra copy, genetic history, and pure chance.

Trisomy 21 is usually caused by nondisjunction in the gametes prior to conception, and all cells in the body are affected. However, when some of the cells in the body are normal and other cells have trisomy 21, it is called mosaic Down syndrome (46,XX/47,XX,+21). This can occur in one of two ways: a nondisjunction event during an early cell division in a normal embryo leads to a fraction of the cells with trisomy 21; or a Down syndrome embryo undergoes nondisjunction and some of the cells in the embryo revert to the normal chromosomal arrangement. There is considerable variability in the fraction of trisomy 21, both as a whole and among tissues. Mosaicism is present in 1-2% of the observed Down syndrome cases.

The incidence of trisomy 21 is estimated at one per 800 to one per 1000 births and increases with maternal age, making it one of the most common chromosomal abnormalities. Pregnant women can be pre-screened late in the first trimester or early second trimester by non-invasive testing procedures that may suggest the presence of a DS fetus, However, these non-invasive tests are not definitive and have a high false positive rate requiring additional testing to verify the diagnosis following a potentially positive screening test result. Definitive testing is accomplished with amniocentesis or chorionic villus sampling (CVS), followed by cell culturing and karyotyping. These procedures provide accurate results, but are labor intensive, expensive, take approximately two weeks to complete, and carry a risk of miscarriage or injury to the fetus.

A novel, rapid, accurate, and safe method of prenatal screening for trisomy 21 is urgently needed in the art. The present invention meets this need.

### SUMMARY OF THE INVENTION

One embodiment of the invention comprises a method of diagnosing chromosomal trisomy in a human subject. The method comprises pyrosequencing at least one single nucleotide polymorphism on a chromosome being assessed for trisomy, where the SNP comprises two alleles. The pyrosequencing method comprising the steps of contacting an isolated DNA sample from the subject with at least one informative primer that specifically binds at a position adjacent to a single nucleotide polymorphism on a chromosome being assessed for trisomy of the subject under conditions suitable for elongation of a nucleic acid complementary to the isolated DNA sample, wherein the number of the expected elongated nucleic acids corresponds to the number of primers that bind to the DNA sample; elongating the nucleic acid complementary to the isolated DNA sample, where incorporation of a deoxynucleotide triphosphate into the complementary strand creates a detectable signal, where the detectable signal represents the presence of one or two alleles; and, detecting the allelic ratio or the relative allele strength (RAS) of the detectable signals of the two alleles, where when the allelic ratio of the two alleles is about 2:1 or the RAS of the two alleles is about 66%:33%, then the subject is diagnosed as having trisomy of the chromosome.

In one aspect, the chromosome being assessed for trisomy is selected from the group consisting of chromosome 21, chromosome 18, chromosome 16, chromosome 13, chromosome 12, chromosome 9, chromosome 8, and any combination thereof. In another aspect, the primers are selected from the group consisting of SEQ ID NO. 1-9. In still another aspect, the human subject is a fetus.

Another embodiment of the invention comprises a kit for diagnosing a chromosomal trisomy in a human subject. The kit comprises at least one primer that specifically binds at a position adjacent to a single nucleotide polymorphism on a chromosome present in an isolated DNA sample obtained from the subject, an applicator, and instructional material for the use thereof. In one aspect, the chromosome being assessed for trisomy is selected from the group consisting of chromosome 21, chromosome 18, chromosome 16, chromosome 13, chromosome 12, chromosome 9, chromosome 8, and any combination thereof. In another aspect, the primers are selected from the group consisting of SEQ ID NO. 1-9. In still another aspet, the human is a fetus.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic diagram depicting the location of nine single nucleotide polymorphism (SNP) markers spanning the q-arm of chromosome 21, labeled 1-9 by the arrows to the right of the diagram.

FIG. 2, is a series of graphs depicting pyrograms showing that it is possible to distinguish trisomy 21 from normal genotype using a single chromosome 21 marker. Peaks on each pyrogram correlate with intensity of a single nucleotide signal at each position shown. Dashed bar shows intensity for one allele (C containing); solid bar shows intensity for the other allele (T containing). Y-axis shows signal intensity; X-axis is identity of the nucleotide added in each cycle of the Pyrosequencing reaction.

### DETAILED DESCRIPTION OF THE INVENTION

The present invention is based in part on the discovery of a rapid, selective, and accurate method of detecting chromosomal trisomy and trisomy mosaicism in a subject by single nucleotide polymorphism (SNP) genotyping. The invention encompasses compositions, methods, and kits useful in detecting at least one informative chromosomal marker of the invention in a body sample obtained from a subject.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

By the term “applicator” as the term is used herein, is meant any device including, but not limited to, a hypodermic syringe, a pipette, a buccal swab, and other means for using the kits of the present invention.

As used herein, an “allele” is one of several alternate forms of a gene or non-coding regions of DNA that occupy the same position on a chromosome.

“Biological sample,” as that term is used herein, means a sample obtained from a subject, preferably a mammal, that can be used as a source to obtain nucleic acid from that subject.

The phrase “body sample” as used herein, is intended any sample comprising a cell, a tissue, or a bodily fluid in which chromosomal material can be detected. Samples that are liquid in nature are referred to herein as “bodily fluids.” Body samples may be obtained from a patient by a variety of techniques including, for example, by scraping or swabbing an area or by using a needle to aspirate bodily fluids. In one embodiment, the body sample may be fluid obtained from a pregnant female, including saliva, urine, blood, or amniotic fluid. A body sample may also include cells or tissue obtained from a fetus. As an example, for prenatal diagnosis of chromosomal trisomy, a biological sample of amniotic fluid, chorionic villous biopsy, fetal cells in maternal circulation, fetal blood cells extracted from an umbilical artery or vein, fetal cells from premortem or postmortem tissues, and fixed tissue can be used in the methods of the present invention. Methods for collecting such biological samples from a mother or a fetus are well known in the art and include amniocentesis, venous blood draws, and standard histology or pathology techniques.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when corresponding positions in each of the molecules are occupied by nucleotides which normally base pair with each other (e.g., A:T and G:C nucleotide pairs).

“Substantially complementary to” refers to probe or primer sequences which hybridize to the sequences listed under stringent conditions and/or sequences having sufficient homology with test polynucleotide sequences, such that the allele specific oligonucleotide probe or primers hybridize to the test polynucleotide sequences to which they are complimentary.

The term “DNA” as used herein is defined as deoxyribonucleic acid,

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Sequence variation” as used herein refers to any difference in nucleotide sequence between two different oligonucleotide or polynucleotide sequences.

“Polymorphism” as used herein refers to a sequence variation in a gene which is not necessarily associated with pathology.

“Single nucleotide polymorphism” as used herein, is a DNA sequence variation occurring when a single nucleotide (A, T, C, or G) in the genome differs between members of a species, or between paired chromosomes in an individual, and both versions are observed in the general population at a frequency greater than 1%. Almost all common SNPs have only two alleles (by way of a non limiting example one allele may be designated allele “A” and the other allele may be designated allele “B”). Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are nonsynonymous. A nonsynonymous change may either be missense or “nonsense”, where a missense change results in a different amino acid, while a nonsense change results in a premature stop codon. SNPs that are not in protein-coding regions may still have consequences for gene splicing, transcription factor binding, or the sequence of non-coding RNA. Variations in the DNA sequences of humans, e.g. SNPs, can affect how humans develop diseases and respond to pathogens, chemicals, drugs, vaccines, and other agents.

“Mutation” as used herein refers to an altered genetic sequence which results in the gene coding for a non-functioning protein or a protein with substantially reduced or altered function. Generally, a deleterious mutation is associated with pathology or the potential for pathology.

“Allele specific detection assay” as used herein refers to an assay to detect the presence or absence of a predetermined sequence variation in a test polynucleotide or oligonucleotide by annealing the test polynucleotide or oligonucleotide with a polynucleotide or oligonucleotide of predetermined sequence such that differential DNA sequence based techniques or DNA amplification methods discriminate between normal and mutant.

As used herein, an “instructional material” includes a publication, a recording, a diagram, or any other medium of expression, which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container, which contains the nucleic acid, peptide, chemical compound and/or composition of the invention or be shipped together with a container, which contains the nucleic acid, peptide, chemical composition, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Mosaicism” is used herein to refer to a genotype wherein a proportion of cells of an organism have a normal compliment of genes, and a proportion of cells have an abnormal complement of genes.

A “polynucleotide” means a single strand or parallel and anti-parallel strands of a nucleic acid. Thus, a polynucleotide may be either a single-stranded or a double-stranded nucleic acid.

A “portion” of a polynucleotide means at least about fifteen to about fifty sequential nucleotide residues of the polynucleotide. It is understood that a portion of a polynucleotide may include every nucleotide residue of the polynucleotide. “Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

By the term “specifically binds,” as used herein, is meant a primer that recognizes and binds a complementary polynucleotide, but does not recognize and bind other polynucleotides in a sample.

### Description:

The present invention provides compositions, methods, and kits for identifying a subject with chromosomal trisomy.

### I. Compositions

### Nucleic Acids: Target Sequences

The genomic sequences of trisomy 21 markers 1-9 (SEQ ID Nos. 1-9) useful in the methods, assays, and kits of the present invention comprise, but are not limited to those listed in Table 1 below. All nucleotide sequences are listed from the 5′ to 3′ direction.

The target sequence or target nucleic acid may be a portion of a gene, a regulatory sequence, genomic DNA, cDNA, and RNA (including mRNA and rRNA). Genomic DNA samples are usually amplified before being brought into contact with a probe. Genomic DNA can be obtained from any biological sample, including, by way of non-limiting example, tissue source or circulating cells (other than pure red blood cells). For example, convenient sources of genomic DNA include whole blood, semen, saliva, tears, urine, fecal material, sweat, buccal cells, skin and hair. Amplification of genomic DNA containing a polymorphic site generates a single species of target nucleic acid if the individual from which the sample was obtained is homozygous at the polymorphic site, or two species of target molecules if the individual is heterozygous. RNA samples also are often subject to amplification. In this case, amplification is typically preceded by reverse transcription. Amplification of all expressed mRNA can be performed as described in, for example, PCT Publication Nos. WO96/14839 and WO97/01603, which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction. In one embodiment of the invention, the target nucleic acid are SNPs present on the human q-arm of chromosome 21.

### Nucleic Acids: Primers

Table 2 provides primer sequences useful for detecting SNP markers 1-9 in PCR reactions. Table 3 provides extension primers useful in pyrosequencing reactions.

The present invention encompasses isolated nucleic acids useful in the practice of the methods of the invention. Specifically, the present invention encompasses primers useful in the amplification of SNPs located on the q arm of chromosome 21. Each primer should be sufficiently long to initiate or prime the synthesis of extension DNA products in the presence of an appropriate polymerase and other reagents. Appropriate primer length is dependent on many factors, as is well known; typically, in the practice of applicant's method, a primer will be used that contains 15-30 nucleotide residues. Short primer molecules generally require lower reaction temperatures to form and to maintain the primer-template complexes that support the chain extension reaction.

The primers used need to be substantially complementary to the nucleic acid containing the selected sequences to be amplified, i.e, the primers must bind to, i.e. hybridize with, nucleic acid containing the selected sequence (or its complement). The primer sequence need not be entirely an exact complement of the template; for example, a non-complementary nucleotide fragment or other moiety may be attached to the 5′ end of a primer, with the remainder of the primer sequence being complementary to the selected nucleic acid sequence. Primers that are fully complementary to the selected nucleic acid sequence are preferred and typically used.

Generally, primers will be between about 15 and 30 nucleotides in length and preferably between about 18 and 27 nucleotides in length. They are preferably chosen to hybridize to a unique DNA sequence in the genome so as to maximize the desired location hybridization that will occur.

In one embodiment of the invention, the forward primers of the pair of primers that are used preferably have an anchoring moiety covalently linked to the 5′ end of each primer. The reverse primers are derivatized with phosphate at the 5′ ends. Generally, any anchoring moiety can be used that will serve to couple the oligonucleotide to a solid surface or solid phase.

As is well known in this art, various solid phase material can be used; for example, the solid support material can be selected from any of a wide variety of materials that are commonly used, such as those that are commercially available from Amersham Biosciences, BioRad, and Sigma. It can be in the form of particles, plates, matrices, fibers or the like, and it may be made of silica, cellulose, agarose beads, controlled-pore glass, polymeric beads, gel beads, or magnetic beads. Magnetic beads are preferred because the We of such facilitates their subsequent separation from the supernatant by the straightforward application of a magnetic field. Such can be done using flow chambers or by simply pipetting. Such magnetic beads, for example those sold as Dynal beads or those sold by Advanced Magnetics, can be used to separate the amplified DNA from the remainder of the biological sample and the PCR material and reaction products by washing. This same property is also used to advantage in separating decoupled target material, at a later stage in the assay procedure. Although the particles in bead form are preferred for facility and handling, other shaped particles or substrates might alternatively be employed. Such commercially available magnetic beads are generally small non-porous spheres that are coated with a layer of magnetite to provide the desired magnetic properties, and then with an exterior coating. Magnetic beads that are commercially available for these purposes are produced in various ways; often paramagnetic metals, such as metal oxides, are encapsulated with a suitable coating material, such as a polymer or a silicate, to produce coated beads that are about 1 μm-100 μm in diameter.

Anchoring moieties and coupling agents that are complementary and bind to each other are used as a linkage to attach the amplified DNA to such solid support. Many varieties of binding pairs are well known in the art and may be suitably employed. The anchoring moiety may join directly to the solid phase or, more usually, to a complementary coupling agent carried by the solid phase. A preferred binding system employs avidin or streptavidin and biotin. Streptavidin, for example, is covalently attached to the exterior surface of the solid support, e.g., the magnetic beads, and it, in turn, binds strongly to biotinylated DNA. Such magnetic beads suitable for applications of interest are commercially available from a number of vendors. Beads that have streptavidin bound to the surface of the beads, having a nominal size of about 1 micron in diameter, are sold by Active Motif of Carlsbad, Calif. Other binding pairs, e.g. antibody-antigen and the like, may alternatively be used as such an intermediate linkage.

### Nucleic Acids: Synthesis

An isolated nucleic acid of the present invention can be produced using conventional nucleic acid synthesis or by recombinant nucleic acid methods known in the art (2001, Molecular Cloning: A Laboratory Manual, Cold Spring Harbor Laboratory Press, New York) and Ausubel et al. (2001, Current Protocols in Molecular Biology, Green & Wiley, New York).

Tags

In one embodiment of the invention, an isolated nucleic acid of the invention comprises a covalently linked tag. By way of a non-limiting example, an isolated nucleic acid of the present invention may comprise a primer, an oligonucleotide, and a target sequence. That is, the invention encompasses a chimeric nucleic acid wherein the isolated nucleic acid sequence comprises a tag molecule. Such tag molecules are well known in the art and include, for instance, a ULS reagent that reacts with the N-7 position of guanine residues, an amine-modified nucleotide, a 5-(3-aminoallyl)-dUTP, an amine-reactive succinimidyl ester moiety, a biotin molecule, ³³P, ³²P, fluorescent labels such as fluorescein (FITC), 5,6-carboxymethyl fluorescein, Texas Red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7.

However, the invention should in no way be construed to be limited to the nucleic acids encoding the above-listed tags. Rather, any tag that may function in a manner substantially similar to these tag polypeptides should be construed to be included in the present invention.

The isolated nucleic acid comprising a tag can be used to localize an isolated nucleic acid, for example, within a cell, a tissue, and/or a whole organism (e.g., a mammalian embryo), detect an isolated nucleic acid, for example, in a cell, and to study the role(s) of an isolated nucleic acid in a cell. Further, addition of a tag facilitates isolation and purification of the isolated nucleic acid.

TABLE 1
Genomic sequences for each SNP marker.

| | SEQ | |
|---|---|---|
| SNP | ID | |
| Marker | NO. | Genomic Sequence |
| 1 | 1 | TAAAACTAGTCCTACAAGTTTCATGTTTAAAAACCTGTTTATTGAATGTTAACACATTCCATAAGAATAATATC |
| | | CACTTTTAAAAGATATCTGAATTAAGTTGCATGTTTTCATAGCTTTTATTATATGGACATTTATTAGCCCACAG |
| | | CACCCTCAAAAGATCTGAACTTCRAAATCTATGCAGACATTTTCACTCTTTC[A/G]TGCDTAAGGATAAAGTC |
| | | ACACTGTCCTCATTTGGCCACATGTAGTCACTTTTTGAGGGACAATGTGTGGGGGTTGATTTCTACAAAGCAAA |
| | | ATGTAAACATATAATGAAATACATATAAGCCAGATGATGAACAAAACTTCTTACAAATGATAAACAAACAAATG |
| | | TTTGTTGTAATTCATTTTTTCCCTCAATGACAATT |
| 2 | 2 | CCCACATGAATTAGTACCGTGAGAATTTATCTTATATAATTAACATAGCACTTACCTAGAATATATGAATCCTC |
| | | GAACTTATGTGTTAATTCTTGATCTCAATGCTAAGGCTTGAATCTTCAATTCATGTGACTGTTTGATTAATCCT |
| | | CATGAATACTTGACCGTTTTTACAAAATCAATATTTTGACTTTTTGTATCAC[A/G]TGTGTTCTATTCCTTTC |
| | | TGAAATTTCTTAACACAGCTGACAAACACAGGTACAAAGATTTATAGCTTGGGTTCTGAACTGAGCTACTTTGA |
| | | TATGAATCTAAAAAGACATGCCATATTAAAATATGCCTTTAGTCTACRGCCAATTAAAGAAATTAGTGTTAAAA |
| | | GAAGAAATCTGGGTGATTCTGAGATTTAGTTTATA |
| 3 | 3 | CCCACATGAATTAGTACCGTGAGAATTTATCTTATATAATTAACATAGCACTTACCTAGAATATATGAATCCTC |
| | | GAACTTATGTGTTAATTCTTGATCTCAATGCTAAGGCTTGAATCTTCAATTCATGTGACTGTTTGATTAATCCT |
| | | CATGAATACTTGACCGTTTTTACAAAATCAATATTTTGACTTTTTGTATCAC[A/G]TGTGTTCTATTCCTTTC |
| | | TGAAATTTCTTAACACAGCTGACAAACACAGGTACAAAGATTTATAGCTTGGGTTCTGAACTGAGCTACTTTGA |
| | | TATGAATCTAAAAAGACATGCCATATTAAAATATGCCTTTAGTCTACRGCCAATTAAAGAAATTAGTGTTAAAA |
| | | GAAGAAATCGGGTGATTCTGAGATTTAGTTTATA |
| 4 | 4 | TGATACCATTTATTGTCTTATCCAGTTGTATGCCAGATTTCAGAAAACAGCAGAATGAAGTTAACCTGAAGAAT |
| | | TAGTTGTTTGAAAAACCTGCAAAACTTAGCATGAACTTAAATTTTCTCACCTCTGTAAGTTACATTATTTCTTG |
| | | TGATGACACGTACTTAATACACAAATGAAGCGAGCCCATGATAGCTTTTACA[C/T]AGATATTACAAATAAAT |
| | | GTGTTTATAAAGATTTTATGGAACAGTATGGAGAAGTAAAGGAGTTGCTATAACTCAAAGGTATTTTCTATAAG |
| | | TGTCCAGAAAGCAATGTCAATAATTTCCTAGGGCTGGTGGTTAAATCAATGTGAGTGAATGTTATTATTCCCTC |
| | | GTAGAAATATGTTATGCTTTCTACAAAGAACATGT |
| 5 | 5 | TAGAGAGGGCAGACCGGCATGCACTTGTTCAAGCTGGGAATGTCGCCCTGTCAGGAACAGCAGGAATGGCAGCA |
| | | TGCTCTTTGGGTCTGGAGTTCCTCACACTGAGGGAGTTATAATAGCTGTGGGGTTTCCAGGACTGCTCGTGAAG |
| | | ATTTCACTAACCCTGGCTTTGCCCAAGAAGGAGTAAGTGCTTCATGGAAAAG[G/T]CCCTGGAGGCAGAGTCT |
| | | TGGATCCGGGAGCTTCCAATGTTTCTATGAATCTATGCAAACATGGCTTAACTGCTGGCTCAGTTCTTATTGAC |
| | | TTGAGGGCCTCAAGAAAACTCCAGGGAAGAYGCCAGTGAATTAGAGGATCTTTCTCAAAGACTTTGAGATTCTC |
| | | AAAAATCTGATGATGAACTGGAACATGTGACCATT |
| 6 | 6 | TGGACCGGCCAGACCCCTGTGCCGTGAGAGGCGGGGCGGCGGGGCCGTGGGGGCGCTCGCACTCCCAGCTCATC |
| | | GTGGCATGCGCTGACCCGAAAACCACGAGGTAGARGGAATGAGATCACAACATTTGTTTGCGTTGTCTAAAATT |
| | | ATCCTCTGATTTCATTCCGTGCCTGCGTCAGGAGGGAGAAACATGGGAAGGT[C/T]GTTTGTCTTGGGCAGGG |
| | | AAAGCATCACAAGGGCGCGTTGTGTGTCTGGCTTACCGTCTCTGGACCAAAGCTGTGTTTGTTTTTCTTATCTA |
| | | CCAGTTCCAGTAAGCCAAACCTCTTGGCGTGGGTTTCCTTCTGGTTAAGGGGAGGGCTGGCTTCAGAGAGTGAA |
| | | AGACAATAAAAACGTGGAGCTCTGTCCCCTGGCAT |
| 7 | 7 | CCCAGAGGTGGTCTGGGAGCCCTCGCGAGTCAGGCCCTCAATGTCTCCCCTAAATCACTTTGTCAGAATTAGTG |
| | | AAGGCAGAATCTCTGCAGTGAACAAGTTATGTTCTTTTAGAAAATAACACAATGCGGAGGGAATTCTCAAAAAC |
| | | AACCATGCAAGTGGTGGCAGGAGTGGCTGTTGTAGGGGAGGGAGGAGCCTAC[C/T]AAGCAGGGAGGAGGCTG |
| | | GGTGCAGAGGCCTGGCGGGAGGGGACTATGTTCCCAGGTGGCTGACCCAGCTCAGCTCCACGCCCCTGTCCCAT |
| | | GGTCATGCCAGCAGGTGGACCCCAGGGGCTCCAGCTTTATTCTGGGGCCTCTGAGAGCCAGGTCAGCCCTATGT |
| | | CAGCTCCACGMTCTCACTGAGCCATGCACTTACAA |
| 8 | 8 | CTCAGTGGATTGTCTGTRGGAAACTTGCAGCTCTGCTCCTCACACCAGGCCCGGCTGGCCACCCACCCTCGCCC |
| | | CCACTGGCCACCCCKCCCTCGCCCCGACTGCCCCGCCCCACCCTCACCCCGACTGCCCCGCCCTCKCCCGGCTG |
| | | GCCGTCCCTGCCCTCGCCCCGGCTGGCAGGTGCACATGGGGCCTCCAGGTCT[A/G]CCATTCGCTATTGAGAA |
| | | CTAGAAATGAGGAAGGACAGTTACGCTAACTCCAAAAGGCTGTCTAGGATGAGCTGCTTTATCAGGGAGCTCCT |
| | | TGTACCCATTTTACAGAAATCATTTTTAGGTCTTTGTGCCACCACCACGAGGGGCATCTGCAAAGAGGGCAACG |
| | | CTAGACACAGAATCCGTGGAAGGTGCAGCAGTGCC |
| 9 | 9 | GCTGCTTGTGTTGGAGACACAGGCCCAGAGCCACTCCTGCCTACAGGTTCTGAGGGCTCAGGGGACCTCCTGGG |
| | | CCCTCAGGCTCTTTAGCTGAGAATAAGGGCCCTGAGGGAACTACCTGCTTCTCACATCCCCGGGTCTCTGACCA |
| | | TCTGCTGTGTGCCCCGACCCCCCCTACCCTGCTCCTCCACCAAGCCTGATGC[C/T]AAGGGCTATAAACCACT |
| | | GGCCCAACAGAAGCTTGGTTCCCAGAGAACTGGTCCCTGCCTGGGACATGCTCCTTGCTACAGCCCCTTGTGGG |
| | | AGCTCAGAGGGCATGGCTGCTCCCCCTACGGTCCCTCGCCCAGTGGTTCTGTCTCTTTATGGCAGGAAGCAATG |
| | | AGGCTCCCCAAGAACACACCTGAGGAAAAGGACAG |

TABLE 2
PCR Primers

| | SEQ | | SEQ | |
|---|---|---|---|---|
| SNP | ID | PCR | ID | |
| Marker | NO. | primer 1 | NO. | PCR primer 2 |
| 1 | 10 | TGTGGCCAA | 19 | ATTAACCCTCACTAAAGGGAGA |
| | | ATGAGGACA | | CATTTATTAGCCCACAGCACC |
| 2 | 11 | AGTTCAGAACCC | 20 | ATTAACCCTCACTAAAGGGACT |
| | | AAGCTATAAATC | | TGACCGTTTTTACAAAATCAAT |
| 3 | 12 | CTGGGTTGGGTT | 21 | ATTAACCCTCACTAAAGGGATT |
| | | CAGTTTCTTTTA | | TGTACTCAGACCTTCCCCACAG |
| 4 | 13 | CAAATGAAGCGA | 22 | ATTAACCCTCACTAAAGGGACC |
| | | GCCCATGATAG | | ACCAGCCCTAGGAAATTATTGA |
| 5 | 14 | ATAGCTGTGG | 23 | ATTAACCCTCACTAAAGGGAAT |
| | | GGTTTCCAGG | | TGGAAGCTCCCGGATC |
| 6 | 15 | ATTCGGTGC | 24 | ATTAACCCTCACTAAAGGGAAA |
| | | CTGCGTCAG | | GCCAGCCCTCCCCTTAA |
| 7 | 16 | AATGCGGAGG | 25 | ATTAACCCTCACTAAAGGGAAC |
| | | GAATTCTCA | | CTGCTGGCATGACCAT |
| 8 | 17 | CCTGATAAAGCA | 26 | ATTAACCCTCACTAAAGGGAGCT |
| | | GCTCATCCTAG | | GGCAGGTGCACATGG |
| 9 | 18 | TCCTCCACCA | 27 | ATTAACCCTCACTAAAGGGACTT |
| | | AGCCTGATG | | CTGTTGGGCCAGTGGTTTAT |

TABLE 3
Pyrosequencing extension primer sequences.

| SNP | SEQ ID | Pyrosequencing Extension |
|---|---|---|
| marker | NO. | Primer |
| 1 | 28 | ACAGTGTGACTTTATCCTTA |
| 2 | 29 | TTTCAGAAAGGAATAGAACA |
| 3 | 30 | CCAATGAAACCATCCT |
| 4 | 31 | GCCCATGATAGCTTT |
| 5 | 32 | AGTGCTTCATGGAAAAG |
| 6 | 33 | GAGAAACATGGGAAGGT |
| 7 | 34 | GGAGGGAGGAGCCTAC |
| 8 | 35 | AGTTCTCAATAGCGAATG |
| 9 | 36 | CACCAAGCCTGATGC |

### II. Methods

The present invention includes a method of screening and diagnosing a subject for chromosomal trisomy using at least one single nucleotide polymorphism (SNP) marker present on a chromosome of interest. The present invention further comprises a method of screening for and diagnosing a subject for chromosomal trisomy mosaicism using at least one SNP present on a chromosome of interest. A skilled artisan will appreciate, however, that it may be desirable to use more than one informative SNP marker. Accordingly, in one embodiment, the method of the invention encompasses using a panel of informative markers comprising at least two, at least three, at least four, at least five, at least 6, at least seven, at least eight, at least nine, at least 10 or more informative SNP markers distributed along the length of the chromosome being assessed for trisomy.

The method comprises isolating a nucleic acid sample from a body sample obtained from a subject and screening the nucleic acid sample for at least one chromosomal trisomy using a panel of informative markers specific for SNPs present on the chromosome. If a chromosomal trisomy is detected in the nucleic acid sample, then the subject is identified as having trisomy of that chromosome.

A nucleic acid sample is any type of nucleic acid sample in which potential SNPs exist. For instance, the nucleic acid sample may be an isolated genome or a portion of an isolated genome. An isolated genome consists of all of the DNA material from a particular organism, i.e., the entire genome. A portion of an isolated genome, which is referred to as a reduced complexity genome (RCG), is a plurality of DNA fragments within an isolated genome but which does not include the entire genome. Genomic DNA comprises the entire genetic component of a species excluding, when applicable, mitochondrial DNA.

The method may be practiced on a subject, preferably a mammal, more preferably a human. In one embodiment, the subject is a pregnant woman. In another embodiment, the subject is a fetus. A body sample of the invention may be obtained from a subject at an appropriate period of pregnancy. Preferably, the body sample is obtained from a subject during the first or second trimesters of pregnancy.

In one embodiment of the invention, a panel of informative single nucleotide polymorphism (SNP) markers that span at least one chromosome of interest is used in a pyrosequencing assay suitable for quantitative assessment of signal strength from single nucleotides.

Each SNP is a two allele system, with the two alleles designated, for examplary purposes only herein, allele “A” and allele “B”. In a normal individual which is heterozygous for a given SNP marker, a balanced A/B allel ratio is observed and relative allele strength (RAS) is about A50%/B50%. In a normal individual which is homozygous for a given SNP marker, i.e. only allele A or allele B is present, then the RAS would be about A100%/B0% or about A0%/B100%, respectively. If an individual has one extra copy of an entire or a portion of a chromosome, markers spanning the extra chromosome region would show a gain of one allele. This event increases the signal intensity of one allele over the other at a given SNP. By way of a non-limiting example, if three copies of a chromosome are present resulting in an A/B allelic ratio of about 2:1, then a relative allele strength (RAS) of about A66%/B33% would be observed.

A skilled artisan will readily appreciate that any chromosome can be affected by trisomy. However, the most common incidences of chromosomal trisomy affect chromosomes 21, 18, 16, 13, 12, 9, and 8.

In a preferred embodiment, at least one informative single nucleotide polymorphism (SNP) marker for chromosome 21 is used in a pyrosequencing assay suitable for simultaneous qualitative assessment of allele heterozygosity and quantitative assessment of allele signal strength from at least one single nucleotide polymorphism (SNP) on chromosome 21 and present in a nucleic acid sample obtained from a body sample of a subject. If the RAS is either about A50%/B50%, about A100%/B0% or about A0%/B100%, then the subject does not have trisomy 21. If the RAS is about A66%/B33%, then the subject does have trisomy 21. The method of the invention encompasses the use of at least one informative SNP marker present on the 21′ chromosome. A skilled artisan will appreciate it may be desirable to use more than one informative SNP marker distributed along the length of chromosome 21. As demonstrated by the data disclosed herein, the panel of informative markers disclosed elsewhere herein is designed and used to genotype a 47,XX,+21 individual, a 47,XY,+21 individual, as well as an individual with mosaicism, i.e. 46,XX/47,XX,+21 or 46,XY/47,XY,+21.

In another embodiment, at least one informative single nucleotide polymorphism (SNP) marker for chromosome 18 is used in a pyrosequencing assay suitable for simultaneous qualitative assessment of allele heterozygosity and quantitative assessment of allele signal strength from at least one single nucleotide polymorphism (SNP) on chromosome 18 and present in a nucleic acid sample obtained from a body sample of a subject. If the RAS is either about A50%/B50%, about A100%/B0% or about A0%/B100%, then the subject does not have trisomy 18. If the RAS is about A66%/B33%, then the subject does have trisomy 18. The method of the invention encompasses the use of at least one informative SNP marker present on the 18th chromosome. A skilled artisan will appreciate it may be desirable to use more than one informative SNP marker distributed along the length of chromosome 18.

In yet another embodiment, at least one informative single nucleotide polymorphism (SNP) marker for chromosome 16 is used in a pyrosequencing assay suitable for simultaneous qualitative assessment of allele heterozygosity and quantitative assessment of allele signal strength from at least one single nucleotide polymorphism (SNP) on chromosome 16 and present in a nucleic acid sample obtained from a body sample of a subject. If the RAS is either about A50%/B50%, about A100%/B0% or about A0%/B100%, then the subject does not have trisomy 16. If the RAS is about A66%/B33%, then the subject does have trisomy 16. The method of the invention encompasses the use of at least one informative SNP marker present on the 16th chromosome. A skilled artisan will appreciate it may be desirable to use more than one informative SNP marker distributed along the length of chromosome 16.

In still another embodiment, at least one informative single nucleotide polymorphism (SNP) marker for chromosome 13 is used in a pyrosequencing assay suitable for simultaneous qualitative assessment of allele heterozygosity and quantitative assessment of allele signal strength from at least one single nucleotide polymorphism (SNP) on chromosome 13 and present in a nucleic acid sample obtained from a body sample of a subject. If the RAS is either about A50%/B50%, about A100%/B0% or about A0%/B100%, then the subject does not have trisomy 13. If the RAS is about A66%/B33%, then the subject does have trisomy 13. The method of the invention encompasses the use of at least one informative SNP marker present on the 13th chromosome. A skilled artisan will appreciate it may be desirable to use more than one informative SNP marker distributed along the length of chromosome 13.

In yet another embodiment, at least one informative single nucleotide polymorphism (SNP) marker for chromosome 12 is used in a pyrosequencing assay suitable for simultaneous qualitative assessment of allele heterozygosity and quantitative assessment of allele signal strength from at least one single nucleotide polymorphism (SNP) on chromosome 12 and present in a nucleic acid sample obtained from a body sample of a subject. If the RAS is either about A50%/B50%, about A100%/B0% or about A0%/B100%, then the subject does not have trisomy 12. If the RAS is about A66%/B33%, then the subject does have trisomy 12. The method of the invention encompasses the use of at least one informative SNP marker present on the 12th chromosome. A skilled artisan will appreciate it may be desirable to use more than one informative SNP marker distributed along the length of chromosome 12.

In still another embodiment, at least one informative single nucleotide polymorphism (SNP) marker for chromosome 9 is used in a pyrosequencing assay suitable for simultaneous qualitative assessment of allele heterozygosity and quantitative assessment of allele signal strength from at least one single nucleotide polymorphism (SNP) on chromosome 9 and present in a nucleic acid sample obtained from a body sample of a subject. If the RAS is either about A50%/B50%, about A100%/130% or about A0%/B100%, then the subject does not have trisomy 9. If the RAS is about A66%/B33%, then the subject does have trisomy 9. The method of the invention encompasses the use of at least one informative SNP marker present on the 9th chromosome. A skilled artisan will appreciate it may be desirable to use more than one informative SNP marker distributed along the length of chromosome 9.

In another embodiment, at least one informative single nucleotide polymorphism (SNP) marker for chromosome 8 is used in a pyrosequencing assay suitable for simultaneous qualitative assessment of allele heterozygosity and quantitative assessment of allele signal strength from at least one single nucleotide polymorphism (SNP) on chromosome 8 and present in a nucleic acid sample obtained from a body sample of a subject. If the RAS is either about A50%/B50%, about A100%/B0% or about A0%/B100%, then the subject does not have trisomy 8. If the RAS is about A66%/B33%, then the subject does have trisomy 8. The method of the invention encompasses the use of at least one informative SNP marker present on the 8th chromosome. A skilled artisan will appreciate it may be desirable to use more than one informative SNP marker distributed along the length of chromosome 8.

Any method of allele specific sequencing may be used in the practice of this invention, including, but not limited to fluorescence detection, DNA sequencing gel, capillary electrophoresis on an automated DNA sequencing machine, microchannel electrophoresis, and other methods of sequencing, Sanger dideoxy sequencing, dye-terminator sequencing, mass spectrometry, time of flight mass spectrometry, quadrupole mass spectrometry, magnetic sector mass spectrometry, electric sector mass spectrometry infrared spectrometry, ultraviolet spectrometry, palentiostatic amperometry or by DNA hybridization techniques including Southern Blot, Slot Blot, Dot Blot, and DNA microarray, wherein DNA fragments would be useful as both “probes” and “targets,” ELISA, fluorimetry, fluorescence polarization, Fluorescence Resonance Energy Transfer (FRET), SNP-IT, Gene Chips, HuSNP, BeadArray, amplification assays, TaqMan assay, Invader assay, MassExtend, or MassCleave™ (hMC) method.

A preferred method of allele specific sequencing useful ni the practice of the invention is pyrosequencing. Pyrosequencing is a method of DNA sequencing (determining the order of nucleotides in DNA) based on the “sequencing by synthesis” principle, which relies on detection of pyrophosphate release on nucleotide incorporation rather than chain termination with dideoxynucleotides (Ahmadian et al., 2000, Anal. Biochem, 280:103-110; Alderborn et al., 2000, Genome Res. 10:1249-1258 and Fakhrai-Rad et al., 2002, Hum. Mutat. 19:479-485; Margulies, et al., 2005, Nature 437:376-380; Ronaghi et al., 1996, Analytical Biochemistry 242:84-89).

Pyrosequencing comprises a series of steps for the accurate and qualitative analysis of DNA sequences. Pyrosequencing comprises hybridizing a sequencing primer to a single stranded, PCR amplified, DNA template, and incubating the primers and DNA template with the standard PCR enzymes (e.g. DNA polymerase) with ATP sulfurylase, luciferase and apyrase, and the substrates, adenosine 5′ phosphosulfate (APS) and luciferin. The first of four deoxyribonucleotide triphosphates (dNTPs) is added to the reaction as a second step. DNA polymerase catalyzes the incorporation of the deoxyribo-nucleotide triphosphate to the complementary base in the target DNA template strand. Each incorporation event is accompanied by release of pyrophosphate (PPi) in a quantity equimolar to the amount of incorporated nucleotide. In the third step, ATP sulfurylase quantitatively converts PPi to ATP in the presence of APS. This ATP drives the luciferase mediated conversion of luciferin to oxyluciferin and generates visible light proportional to the amount of ATP. The light produced in the luciferase-catalyzed reaction is detected by a charge coupled device (CCD) camera and seen as a peak in a Pyrogram™. The height of each peak (light signal) is proportional to the number of nucleotides incorporated. As a fourth step, apyrase, a nucleotide degrading enzyme, continuously degrades ATP and unincorporated dNTPs. This reaction switches off the light and regenerates the reaction solution. The next dNTP is then added one at a time and the process is repeated for each dNTP (i.e. dCTP, dGTP, dTTP) in the fifth step. Deoxyadenosine alfa-thio triphosphate (dATPaS) is used as a substitute for deoxyadenosine triphosphate (dATP) since it is efficiently used by the DNA polymerase, but not recognized by the luciferase. As the process continues, the complementary DNA strand is built up and the nucleotide sequence is determined from the signal peaks in the Pyrogram. Pyrosequencing analytical software assigns both genotype and quantifies the signal strength of each allele. Genotype and signal strength are outputted to standard spreadsheet format. Methods for accomplishing pyrosequencing reactions are well known in the art and are described in, for example, U.S. Pat. Nos. 6,258,568 and 6,258,568. Kits, apparatuses and reagents for pyrosequencing are commercially available from, for example, Biotage Ab, Uppsala, Sweden).

The method of the present invention comprises contacting a nucleic acid sample obtained from the body sample of a subject with a primer that specifically binds at a position adjacent, or immediately adjacent, to an SNP on a chromosome of interest under conditions suitable for elongation of a nucleic acid complementary to the isolated DNA sample. Conditions suitable for elongation of a complementary nucleic acid are similar or identical to those used for PCR reactions and are described elsewhere herein. In addition, suitable conditions are described in the manufacturer's protocol for pyrosequencing machines (Biotage AB, Uppsala, Sweden).

The number of elongated nucleic acids is identical to the number of primers that bind to the template. The complementary nucleic acid is elongated as described for the pyrosequencing reaction described elsewhere herein. The incorporation of each deoxynucleotide triphosphate into the complementary strand creates a detectable signal (e.g. light). The presence of a detectable signal is captured by a camera and converted into a signal that represents a given allele.

The presence of mosaicism is evaluated and assessed by determining the ratio of signal strength from each 2-allele system for every SNP marker, Assuming a normal distribution around the mean, ratios that differ from 50% or 100% by 0.5 standard deviations (SD) are suggestive of chromosome mosaicism and are flagged as such. As an example, a heterozygote genotype should have equal, or 50% signal from each allele. If one allele provides <27% or >72% of the total signal then mosaicism is possible. If this occurs in at least two of the SNP markers then mosaicism is likely. In the case of a homozygote genotype, then 100% of the signal should come from a single allele. If less than 85% of the signal comes from one allele, then mosaicism is possible.

### III. Kits

The invention encompasses various kits relating to screening, identifying and/or diagnosing chromosomal trisomy in a subject. The kits of the present invention can be used to perform population screening or individual screening of a newborn, a fetus, or a child. The kit of the present invention can comprise primers that specifically bind to chromosome markers disclosed elsewhere herein for diagnosis of chromosomal trisomy, preferably trisomy 21, in various clinical labs. The present invention further comprises kits for the collection of a biological sample. A patient or practitioner can collect a biological sample and send the sample to a clinical lab where the present screen for Turner syndrome is performed.

The present invention further comprises DNA collection kits for detecting chromosomal trisomy. The kits of the present invention can comprise reagents and materials to expedite the collection of samples for DNA extraction and analysis. These kits can comprise an intake form with a unique identifier, such as a bar-code, a sterile biological collection vessel, such as a Catch-All™ swab (Epicentre® Madison, Wis.) for collecting loose epithelial cells from inside the cheek; and an instruction material that depicts how to properly apply the swab, dry it, repack it and return to a clinical lab. The kit can further comprise a return postage-paid envelope addressed to the clinical lab to facilitate the transport of biological samples.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Development of PCR/Pyrosequencing Based Approach for Ds Screening

To detect chromosome 21 trisomy, a novel, pyrosequencing-based method that interrogates vastly more markers than previously developed methods was developed. The approach involves simultaneous qualitative assessment of allele heterozygosity and quantitative assessment of allele signal from a panel of SNP markers spanning chromosomes 21.

### Development of a Panel of SNP Markers

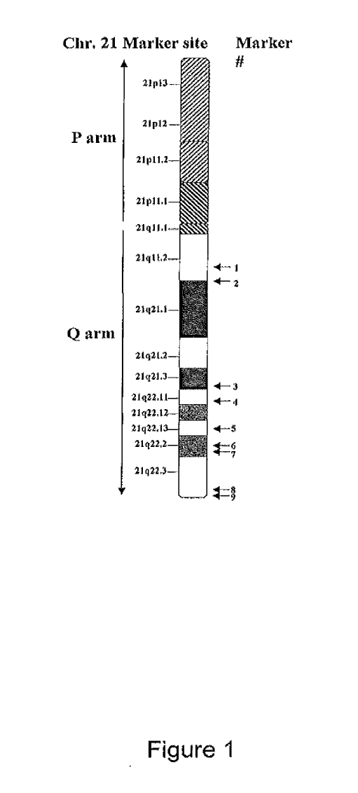

Computer analysis was performed to identify regions of chromosome 21 with high heterozygosity from the publicly accessible non-proprietary Human Genome Resource Database (National Center for Biotechnology Information http://www.ncbi.nlm.nih.gov/genome/guide/human/). Regions with heterozygosity scores greater than 25% were identified, and selected to span part of the q-arm of chromosome 21 from 21q21 to 21q22 (FIG. 1) which includes the Down Syndrome Critical Region. This approach resulted in the initial identification of 40 markers, with 9 markers favorable for testing due to the high heterozygosity value of around 50% and a consistent relative allele strength (RAS) value close to 50% in euploid heterozygote controls (see next section). These markers are comprised of a variable nucleotide (a polymorphism) within a short segment of genomic sequence (generally less than 300 bases in length) present only once in the entire human genome.

The results of the experiments presented in this Example are now described.

### Example 1

### Assessment of Specificity of Markers

To begin to assess the utility of pyrosequencing for interrogation of relative allele strength (RAS), the variance and specificity of each marker on DNA was assessed from 30 individuals without trisomy 21 (46 XX or XY, normal controls). DNAs from the NIGMS Diversity Panel were obtained from the human genetic cell repository of the National Institute of General Medical Sciences (NIGMS/NIH) maintained at the Coriell Institute for Medical Research (Camden, N.J.).

TABLE 4
Relative allele strength (RAS) in normal individuals
for nine chromosome 21 markers.

| | A/B Allele | | |
|---|---|---|---|
| Chr. 21 Marker | (RAS) | 1 SD | 3 SD |
| 1 | 51.6% | 2.0 | 5.9 |
| 2 | 50.2% | 1.3 | 3.9 |
| 3 | 54.0% | 1.3 | 3.9 |
| 4 | 48.8% | 1.6 | 4.8 |
| 5 | 52.8% | 2.0 | 6.1 |
| 6 | 58.2% | 1.4 | 4.1 |
| 7 | 57.6% | 2.1 | 6.3 |
| 8 | 50.7% | 2.5 | 7.5 |
| 9 | 56.9% | 3.7 | 11.2 |

To assess both qualitative heterozygosity and quantitative signal from polymorphic alleles at each SNP marker, genotyping was performed by pyrosequencing. Small segments (50 to 500 base pairs) of genomic DNA were amplified by PCR using oligonucleotide pairs complementary to unique non-proprietary sequence (dbSNP database) flanking the 9 chromosome 21 SNP markers. The pyrosequencing analytical software (PSQ 96MA SNP Software) was then used to quantify the signal strength of each allele and assess genotype. Genotype and signal strength were then exported using a standard spreadsheet format, and compared with the known genotype.

The relative allele strength (RAS) was determined for each marker as related to three possibilities: A+B alleles present equally (A50%/B50%), only A allele present (A100%; B0%); only B allele present (A0%; B100%). Results obtained using the markers in normal euploid controls are shown in Table 1. For each of the markers, two alleles were detected in all individuals. The RAS values for each marker are shown in the second column, and in general were very close to 50% for all markers. The first (column 3) and the third (column 4) standard deviation (SD) was determined for each marker. RAS scores greater than the third SD from the mean for any particular marker will be flagged as abnormal. Based on these data, 9 informative markers that can identify chromosome 21 SNPs have been identified herein.

### Example 2

### Assessment of Sensitivity for Detecting Trisomy 21

Next, the utility of the marker panels to diagnose trisomy 21 was tested. A collection of DNAs from individuals with trisomy 21 and other chromosome 21 aneuploidy was assembled from the National Institute of General Medical Sciences and National Institute of Aging (Table 2).

PCR reactions and pyrosequencing was performed as above. RAS values were calculated for each marker. RAS values >3 SD from the mean, were considered abnormal.

TABLE 5
RAS values for 9 SNPs on chromosome 21

| | chromosome: | | | | | | | | | |
|---|---|---|---|---|---|---|---|---|---|---|
| | 21q11.2 | 21q21.1 | 21q21.3 | 21q21.11 | 21q21.13 | 21q22.2 | 21q22.2 | 21q22.3 | 21q22.3 | |
| | marker: | | | | | | | | | |
| | 1 | 2 | 3 | 4 | 5 | 6 | 7 | 8 | 9 | |
| | SNP: | | | | | | | | | |
| | C/T | C/T | C/T | C/T | C/T | C/T | C/T | G/C | C/T | |
| KARYOTYPE | % T | % T | % T | % T | % T | % T | % T | % G | % T | ETHNICITY |
| GM02504 47XX, +21 | 62.7 | 33.4 | 35.4 | 37.5 | 65.0 | 44.6 | 72.8 | 100.0 | 43.0 | African American |
| GM02571 48XX, +21, +mar | 36.5 | 30.9 | 36.8 | 62.7 | 40.8 | 9.9 | 100.0 | 67.6 | 100.0 | Caucasian |
| AG05121 47XX, +21 | 63.2 | 63.8 | 67.9 | 36.6 | 0.0 | 9.2 | 100.0 | 67.8 | 100.0 | N/A |
| AG05397 47XX, +21 | 33.7 | 25.8 | 36.8 | 34.1 | 66.5 | 44.5 | 74.2 | 69.5 | 38.9 | Caucasian |
| GM01921 47XY, +21 | 62.5 | 65.4 | 67.5 | 88.6 | 40.4 | 71.4 | 38.4 | 42.6 | 73.3 | Caucasian |
| GM02067 47XY, +21 | 90.9 | 100.0 | 100.0 | 100.0 | 34.6 | 0.0 | 100.0 | 0.0 | 100.0 | Caucasian |
| GM02767 47XX, +21 | 36.1 | 32.7 | 0.0 | 33.9 | 38.5 | 72.4 | 89.3 | 73.2 | 71.8 | Caucasian |
| GM04592 47XX, +21 | 62.5 | 63.2 | 68.3 | 35.9 | 67.6 | 0.0 | 100.0 | 72.7 | 100.0 | Caucasian |
| NA17001 | 100.0 | 49.7 | 56.7 | 50.5 | 0.0 | 58.2 | 93.3 | 51.7 | 100.0 | Northern European |
| NA17002 | 100.0 | 91.7 | 100.0 | 0.0 | 0.0 | 60.4 | 100.0 | 49.6 | 100.0 | Northern European |
| NA17003 | 6.4 | 0.0 | 54.5 | 90.5 | 53.5 | 55.8 | 100.0 | 50.7 | 100.0 | Northern European |
| NA17004 | 100.0 | 0.0 | 100.0 | 50.7 | 53.6 | 8.2 | 59.8 | 49.7 | 100.0 | Northern European |
| NA17005 | 0.0 | 0.0 | 0.0 | 92.3 | 0.0 | 0.0 | 56.9 | 0.0 | 52.7 | Northern European |
| NA17006 | 52.6 | 51.6 | 54.2 | 0.0 | 53.5 | 59.0 | 56.2 | 0.0 | 0.0 | Northern European |
| NA17007 | 53.6 | 48.7 | 100.0 | 47.1 | 0.0 | 57.9 | 100.0 | 0.0 | 54.1 | Northern European |
| NA17008 | 50.6 | 51.5 | 0.0 | 100.0 | 0.0 | 58.6 | 100.0 | 54.0 | 100.0 | Northern European |
| NA17009 | 0.0 | 0.0 | 0.0 | 49.0 | 0.0 | 59.3 | 100.0 | 0.0 | 100.0 | Northern European |
| NA17010 | 0.0 | 0.0 | 51.7 | 92.1 | 0.0 | 58.2 | 58.3 | 0.0 | 54.3 | Northern European |
| NA16654 | 52.5 | 51.2 | 100.0 | 49.8 | 7.1 | 59.6 | 100.0 | 51.5 | 100.0 | Chinese |
| NA16688 | 49.5 | 49.7 | 53.0 | 0.0 | 55.4 | 7.1 | 58.3 | 52.3 | 0.0 | Chinese |
| NA16689 | 54.6 | 50.7 | 100.0 | 100.0 | 100.0 | 0.0 | 100.0 | 49.8 | 64.4 | Chinese |
| NA17014 | 50.2 | 60.9 | 94.3 | 48.1 | 51.5 | 54.9 | 100.0 | 0.0 | 58.7 | Chinese |
| NA17015 | 51.3 | 51.1 | 0.0 | 100.0 | 53.3 | 0.0 | 59.1 | 100.0 | 63.3 | Chinese |
| NA17016 | 49.5 | 49.5 | 52.9 | 0.0 | 49.2 | 0.0 | 100.0 | 0.0 | 56.1 | Chinese |
| NA17017 | 53.6 | 52.0 | 100.0 | 49.7 | 50.0 | 58.9 | 55.4 | 50.3 | 100.0 | Chinese |
| NA17018 | 46.9 | 48.3 | 52.8 | 46.5 | 0.0 | 58.9 | 61.4 | 49.4 | 59.1 | Chinese |
| NA17019 | 51.9 | 48.6 | 54.6 | 0.0 | 100.0 | 57.6 | 55.6 | 100.0 | 57.3 | Chinese |
| NA17020 | 100.0 | 93.5 | 54.1 | 51.4 | 53.3 | 100.0 | 100.0 | 100.0 | 100.0 | Chinese |
| NA17031 | 0.0 | 48.0 | 100.0 | 46.6 | 53.1 | 0.0 | 100.0 | 51.4 | 100.0 | African American |
| NA17032 | 52.4 | 51.3 | 54.6 | 48.8 | 57.1 | 59.0 | 0.0 | 45.7 | 100.0 | African American |
| NA17033 | 51.8 | 0.0 | 54.3 | 0.0 | 0.0 | 57.3 | 57.7 | 51.8 | 52.8 | African American |
| NA17034A | 0.0 | 0.0 | 51.9 | 50.6 | 51.0 | 0.0 | 100.0 | 56.2 | 57.8 | African American |
| NA17035A | 0.0 | 50.6 | 100.0 | 0.0 | 100.0 | 8.8 | 100.0 | 53.1 | 100.0 | African American |
| NA17036 | 100.0 | 100.0 | 100.0 | 49.8 | 52.1 | 8.3 | 53.9 | 100.0 | 54.1 | African American |
| NA17037 | 0.0 | 0.0 | 53.7 | 47.0 | 100.0 | 9.2 | 0.0 | 49.7 | 100.0 | African American |
| NA17038 | 50.6 | 0.0 | 55.5 | 46.7 | 0.0 | 58.0 | 100.0 | 100.0 | 54.7 | African American |
| NA17039 | 53.6 | 0.0 | 54.0 | 48.7 | 100.0 | 6.5 | 0.0 | 47.9 | 7.7 | African American |
| NA17040A | 0.0 | 0.0 | 55.1 | 47.9 | 52.4 | 100.0 | 58.9 | 46.9 | 100.0 | African American |
| PCR Blank | 0.0 | 0.0 | 0.0 | 0.0 | 0.0 | 0.0 | 0.0 | 0.0 | 0.0 | |

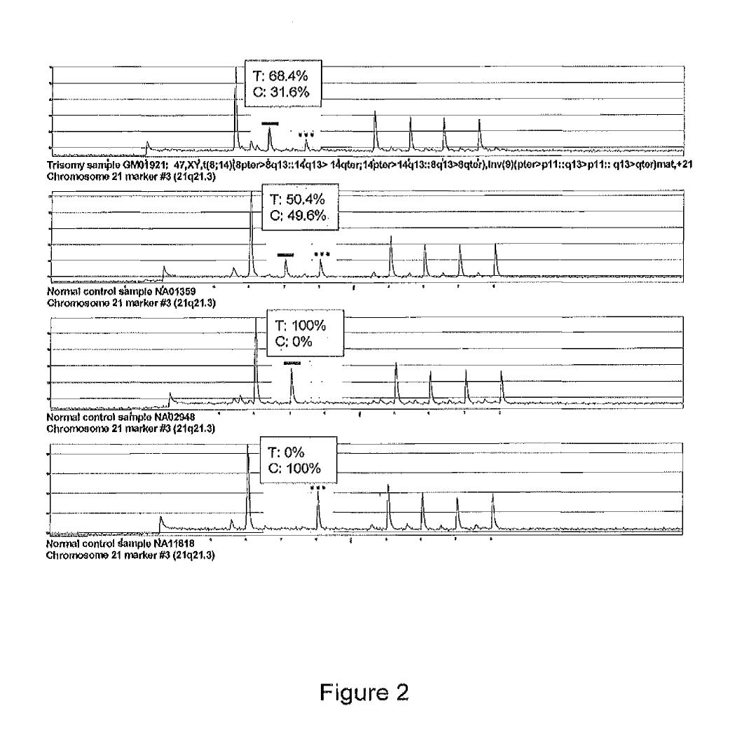

If an individual has one extra copy of an entire or a portion of chromosome 21, markers spanning the extra chromosome region showed a gain of one allele. This event increased the signal intensity of one allele over the other, resulting in an A/B allelic ratio of about 2:1 or RAS values of about 66.6%/33.3% (FIG. 2). The method of the invention could detect extra chromosome 21 alleles in each of the four individuals with DS. (Table 6; FIG. 2).

TABLE 6
Coriell cell lines genomic DNA from individual with Trisomy 21

| Cell Lines | Cytogenetic Diagnosis |
|---|---|
| GM01921 | 47, XY, t(8; 14)(8pter > 8q13::14q13> |
| | 14qter; 14pter > 14q13::8q13 > 8qter), |
| | inv(9)(pter > p11::q13 > p11::q13 > qter)mat, +21 |
| GM02067 | 47, XY, +21 |
| GM02767 | 47, XY, +21 |
| GM04592 | 47, XY, +21 |
| GM02504 | 47, XX, +21 |
| GM02571 | 48, XX, +21, +mar |
| AG05121 | 47, XX, +21 |
| AG05397 | 47, XX, +21 |

Collectively, these data suggests that it is possible to develop a pyrosequencing-based method for the detection of DS and expertise to develop a SNP-based trisomy 21 screening. These results suggest that high-throughput and low cost screening for trisomy 21 using quantitative and qualitative genotyping by pyrosequencing is feasible.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of diagnosing chromosomal trisomy in a human subject, said method comprising pyrosequencing at least one single nucleotide polymorphism on a chromosome being assessed for trisomy, wherein said SNP comprises two alleles, said method of pyrosequencing comprising the steps of:
a) contacting an isolated DNA sample from said subject with at least one informative primer that specifically binds at a position adjacent to a single nucleotide polymorphism on a chromosome being assessed for trisomy of said subject under conditions suitable for elongation of a nucleic acid complementary to said isolated DNA sample, wherein the number of said expected elongated nucleic acids corresponds to the number of primers that bind to the DNA sample;
b) elongating said nucleic acid complementary to said isolated DNA sample, wherein incorporation of a deoxynucleotide triphosphate into said complementary strand creates a detectable signal, wherein said detectable signal represents the presence of one or two alleles; and,
e) detecting the allelic ratio or the relative allele strength (RAS) of said detectable signals of the two alleles, wherein when the allelic ratio of the two alleles is about 2:1 or the RAS of the two alleles is about 66%:33%, then said subject is diagnosed as having trisomy of said chromosome.

2. The method of claim 1, wherein the chromosome being assessed for trisomy is selected from the group consisting of chromosome 21, chromosome 18, chromosome 16, chromosome 13, chromosome 12, chromosome 9, chromosome 8, and any combination thereof.

3. The method of claim 1, wherein said primers are selected from the group consisting of SEQ ID NO. 1-9.

4. The method of claim 1, wherein said human subject is a fetus.

5. A kit for diagnosing a chromosomal trisomy in a human subject, said kit comprising at least one primer that specifically binds at a position adjacent to a single nucleotide polymorphism on a chromosome present in an isolated DNA sample obtained from said subject, an applicator, and instructional material for the use thereof.

6. The kit of claim 5, wherein the chromosome being assessed for trisomy is selected from the group consisting of chromosome 21, chromosome 18, chromosome 16, chromosome 13, chromosome 12, chromosome 9, chromosome 8, and any combination thereof.

7. The kit of claim 5, wherein said primers are selected from the group consisting of SEQ ID NO. 1-9.

8. The kit of claim 7, wherein said human is a fetus.


SEQUENCE LISTING (36 sequences)

SEQ ID NO: 1 - DNA, 402 bp, Artificial Sequence
  chemically synthesized
taaaactagtcctacaagtttcatgtttaaaaacctgtttattgaatgttaacacattccataagaataatatccacttttaaaagatatctgaattaagttgcatgttttcatagcttttattatatggacatttattagcccacagcaccctcaaaagatctgaacttcraaatctatgcagacattttcactctttcagtgcdtaaggataaagtcacactgtcctcatttggccacatgtagtcactttttgagggacaatgtgtgggggttgatttctacaaagcaaaatgtaaacatataatgaaatacatataagccagatgatgaacaaaacttcttacaaatgataaacaaacaaatgtttgttgtaattcattttttccctcaatgacaatt

SEQ ID NO: 2 - DNA, 402 bp, Artificial Sequence
  chemically synthesized
cccacatgaattagtaccgtgagaatttatcttatataattaacatagcacttacctagaatatatgaatcctcgaacttatgtgttaattcttgatctcaatgctaaggcttgaatcttcaattcatgtgactgtttgattaatcctcatgaatacttgaccgtttttacaaaatcaatattttgactttttgtatcacagtgtgttctattcctttctgaaatttcttaacacagctgacaaacacaggtacaaagatttatagcttgggttctgaactgagctactttgatatgaatctaaaaagacatgccatattaaaatatgcctttagtctacrgccaattaaagaaattagtgttaaaagaagaaatctgggtgattctgagatttagtttata

SEQ ID NO: 3 - DNA, 402 bp, Artificial Sequence; identical to SEQ ID NO: 2
  chemically synthesized

SEQ ID NO: 4 - DNA, 402 bp, Artificial Sequence
  chemically synthesized
tgataccatttattgtcttatccagttgtatgccagatttcagaaaacagcagaatgaagttaacctgaagaattagttgtttgaaaaacctgcaaaacttagcatgaacttaaattttctcacctctgtaagttacattatttcttgtgatgacacgtacttaatacacaaatgaagcgagcccatgatagcttttacactagatattacaaataaatgtgtttataaagattttatggaacagtatggagaagtaaaggagttgctataactcaaaggtattttctataagtgtccagaaagcaatgtcaataatttcctagggctggtggttaaatcaatgtgagtgaatgttattattccctcgtagaaatatgttatgctttctacaaagaacatgt

SEQ ID NO: 5 - DNA, 402 bp, Artificial Sequence
  chemically synthesized
tagagagggcagaccggcatgcacttgttcaagctgggaatgtcgccctgtcaggaacagcaggaatggcagcatgctctttgggtctggagttcctcacactgagggagttataatagctgtggggtttccaggactgctcgtgaagatttcactaaccctggctttgcccaagaaggagtaagtgcttcatggaaaaggtccctggaggcagagtcttggatccgggagcttccaatgtttctatgaatctatgcaaacatggcttaactgctggctcagttcttattgacttgagggcctcaagaaaactccagggaagaygccagtgaattagaggatctttctcaaagactttgagattctcaaaaatctgatgatgaactggaacatgtgaccatt

SEQ ID NO: 6 - DNA, 402 bp, Artificial Sequence
  chemically synthesized
tggaccggccagacccctgtgccgtgagaggcggggcggcggggccgtggggcgctcgcactcccgagctcatcgtggcatgcgctgagccgaaaaccacgaggtagarggaatgagatcacaacatttgtttgcgttgtctaaaattatcctctgatttcattcggtgcctgcgtcaggagggagaaacatgggaaggtctgtttgtcttgggcagggaaagcatcacaagggcgcgttgtgtgtctggcttaccgtctctggaccaaagctgtgtttgtttttcttatctaccagttccagtaagccaaacctcttggcgtgggtttccttctggttaaggggagggctggcttcagagagtgaaagacaataaaaacgtggagctctgtcccctggcat

SEQ ID NO: 7 - DNA, 402 bp, Artificial Sequence
  chemically synthesized
cccagaggtggtctgggagccctcgcgagtcaggccctcaatgtctcccctaaatcactttgtcagaattagtgaaggcagaatctctgcagtgaacaagttatgttcttttagaaaataacacaatgcggagggaattctcaaaaacaaccatgcaagtggtggcaggagtggctgttgtaggggagggaggagcctacctaagcagggaggaggctgggtgcagaggcctggcgggaggggactatgttcccaggtggctgacccagctcagctccacgcccctgtcccatggtcatgccagcaggtggaccccaggggctccagctttattctggggcctctgagagccaggtcagccctatgtcagctccacgmtctcactgagccatgcacttacaa

SEQ ID NO: 8 - DNA, 402 bp, Artificial Sequence
  chemically synthesized
ctcagtggattgtctgtrggaaacttgcagctctgctcctcacaccaggcccggctggccacccaccctcgcccccactggccacccckccctcgccccgactgccccgccccaccctcaccccgactgccccgccctckcccggctggccgtccctgccctcgccccggctggcaggtgcacatggggcctccaggtctagccattcgctattgagaactagaaatgaggaaggacagttacgctaactccaaaaggctgtctaggatgagctgctttatcagggagctccttgtacccattttacagaaatcatttttaggtctttgtgccaccaccacgaggggcatctgcaaagagggcaacgctagacacagaatccgtggaaggtgcagcagtgcc

SEQ ID NO: 9 - DNA, 402 bp, Artificial Sequence
  chemically synthesized
gctgcttgtgttggagacacaggcccagagccactcctgcctacaggttctgagggctcaggggacctcctgggccctcaggctctttagctgagaataagggccctgagggaactacctgcttctcacatccccgggtctctgaccatctgctgtgtgccccgaccccccctaccctgctcctccaccaagcctgatgcctaagggctataaaccactggcccaacagaagcttggttcccagagaactggtccctgcctgggacatgctccttgctacagccccttgtgggagctcagagggcatggctgctccccctacggtccctcgcccagtggttctgtctctttatggcaggaagcaatgaggctccccaagaacacacctgaggaaaaggacag

SEQ ID NO: 10 - DNA, 18 bp, Artificial Sequence
  chemically synthesized
tgtggccaaatgaggaca

SEQ ID NO: 11 - DNA, 24 bp, Artificial Sequence
  chemically synthesized
agttcagaacccaagctataaatc

SEQ ID NO: 12 - DNA, 24 bp, Artificial Sequence
  chemically synthesized
ctgggttgggttcagtttctttta

SEQ ID NO: 13 - DNA, 23 bp, Artificial Sequence
  chemically synthesized
caaatgaagcgagcccatgatag

SEQ ID NO: 14 - DNA, 20 bp, Artificial Sequence
  chemically synthesized
atagctgtggggtttccagg

SEQ ID NO: 15 - DNA, 18 bp, Artificial Sequence
  chemically synthesized
attcggtgcctgcgtcag

SEQ ID NO: 16 - DNA, 19 bp, Artificial Sequence
  chemically synthesized
aatgcggagggaattctca

SEQ ID NO: 17 - DNA, 23 bp, Artificial Sequence
  chemically synthesized
cctgataaagcagctcatcctag

SEQ ID NO: 18 - DNA, 19 bp, Artificial Sequence
  chemically synthesized
tcctccaccaagcctgatg

SEQ ID NO: 19 - DNA, 43 bp, Artificial Sequence
  chemically synthesized
attaaccctcactaaagggagacatttattagcccacagcacc

SEQ ID NO: 20 - DNA, 44 bp, Artificial Sequence
  chemically synthesized
attaaccctcactaaagggacttgaccgtttttacaaaatcaat

SEQ ID NO: 21 - DNA, 44 bp, Artificial Sequence
  chemically synthesized
attaaccctcactaaagggatttgtactcagaccttccccacag

SEQ ID NO: 22 - DNA, 44 bp, Artificial Sequence
  chemically synthesized
attaaccctcactaaagggaccaccagccctaggaaattattga

SEQ ID NO: 23 - DNA, 38 bp, Artificial Sequence
  chemically synthesized
attaaccctcactaaagggaattggaagctcccggatc

SEQ ID NO: 24 - DNA, 39 bp, Artificial Sequence
  chemically synthesized
attaaccctcactaaagggaaagccagccctccccttaa

SEQ ID NO: 25 - DNA, 38 bp, Artificial Sequence
  chemically synthesized
attaaccctcactaaagggaacctgctggcatgaccat

SEQ ID NO: 26 - DNA, 38 bp, Artificial Sequence
  chemically synthesized
attaaccctcactaaagggagctggcaggtgcacatgg

SEQ ID NO: 27 - DNA, 43 bp, Artificial Sequence
  chemically synthesized
attaaccctcactaaagggacttctgttgggccagtggtttat

SEQ ID NO: 28 - DNA, 20 bp, Artificial Sequence
  chemically synthesized
acagtgtgactttatcctta

SEQ ID NO: 29 - DNA, 20 bp, Artificial Sequence
  chemically synthesized
tttcagaaaggaatagaaca

SEQ ID NO: 30 - DNA, 16 bp, Artificial Sequence
  chemically synthesized
ccaatgaaaccatcct

SEQ ID NO: 31 - DNA, 15 bp, Artificial Sequence
  chemically synthesized
gcccatgatagcttt

SEQ ID NO: 32 - DNA, 17 bp, Artificial Sequence
  chemically synthesized
agtgcttcatggaaaag

SEQ ID NO: 33 - DNA, 17 bp, Artificial Sequence
  chemically synthesized
gagaaacatgggaaggt

SEQ ID NO: 34 - DNA, 16 bp, Artificial Sequence
  chemically synthesized
ggagggaggagcctac

SEQ ID NO: 35 - DNA, 18 bp, Artificial Sequence
  chemically synthesized
agttctcaatagcgaatg

SEQ ID NO: 36 - DNA, 15 bp, Artificial Sequence
  chemically synthesized
caccaagcctgatgc